(19) United States
(12) Patent Application Publication
(10) Pub. No.: US 2025/0333550 A1
(43) Pub. Date: Oct. 30, 2025

(54) PROCESS FOR THE SYNTHESIS OF NEUTRAL POLYMERIC BINDING AGENTS

(71) Applicant: ARIANEGROUP SAS, LES MUREAUX (FR)

(72) Inventors: John EYMANN, VERT LE PETIT (FR); Emilie QUINTARD, VERT LE PETIT (FR); Nicolas VANDECANDELAERE, VERT LE PETIT (FR); Sébastien COMTE, VERT LE PETIT (FR)

(21) Appl. No.: 19/187,316

(22) Filed: Apr. 23, 2025

(30) Foreign Application Priority Data
Apr. 26, 2024 (FR) FR2404267

(51) Int. Cl. C08F 8/32; C08F 220/44

(52) U.S. Cl. CPC C08F 8/32; C08F 220/44; C08F 2400/02

(57) ABSTRACT

A process for the synthesis of a compound of formula (I):

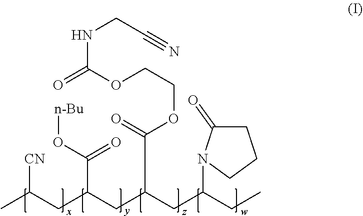

wherein w, x, y and z are as defined in the description.

The process includes reacting a compound of formula (II):

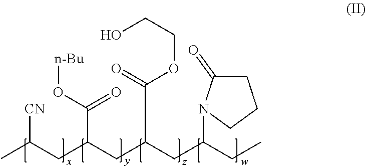

with carbonyldiimidazole, and reacting the resulting compound with propargylamine.

### CROSS-REFERENCE TO RELATED APPLICATIONS

This application claims priority to French Patent Application No. 24 04267, filed Apr. 26, 2024, the entire content of which is incorporated herein by reference in its entirety.

### FIELD

The present disclosure relates to the field of energetic materials, and more particularly to a process for the synthesis of intermediate compounds useful in the preparation of said materials.

### BACKGROUND

Solid composite propellants have a wide range of applications, particularly in the propulsion sector. These propellants are typically prepared from a mixture of fuel, oxidizer and polymer binder. A small amount of bonding agent may be incorporated to guarantee the mechanical properties of these materials. Binding agents of the NPBA (Neutral Polymeric Binding Agents) type are described in U.S. Pat. No. 4,915,755, for example the polymer of formula:

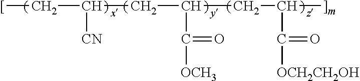

where x′ is 1, y′ is in the range 0-0.5 and z′ is in the range 0.1-0.5.

More recently, Zhou et al. (Iranian Polymer Journal, vol. 28, no. 11, pp. 943-955, 2019) described the synthesis of NPBAs from toluene diisocyanate and propynol ethoxylate. However, the use of diisocyanates is undesirable due to the hazardous nature of these products and their processing.

### SUMMARY OF THE INVENTION

In this context, the inventors set themselves the task of synthesising neutral polymeric binding agents that comply with REACH regulations and are compatible with their use in energetic materials.

The present disclosure relates to a process for the synthesis of a compound of formula (I):

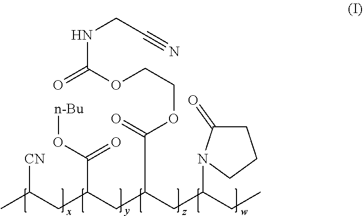

wherein x=1; 0<w≤1; 0<y≤1 and 0<z≤1;
the process comprising:
a) reacting a compound of formula (II):

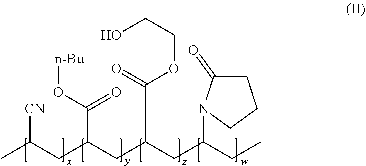

in which, w, x, y and z are as defined above for formula (I),
with an excess of carbonyldiimidazole, optionally in the presence of a solvent;
b) reacting the compound obtained in step a) with an excess of propargylamine, optionally in the presence of a solvent.

The present disclosure also relates to compounds of formula (I) obtainable by the said process.

### DESCRIPTION OF THE INVENTION

In the context of the present disclosure, the expression “between x and y” should be interpreted as including the end values of the range considered (i.e., x and y).

The different embodiments described herein can be combined.

According to one aspect, the present disclosure relates to a process for the synthesis of a compound of formula (I):

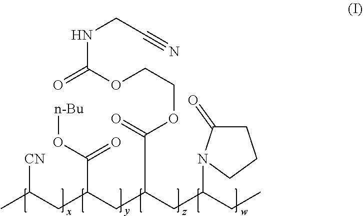

wherein x=1; 0<w≤1; 0<y≤1 and 0<z≤1; the process comprising:
a) reacting a compound of formula (II):

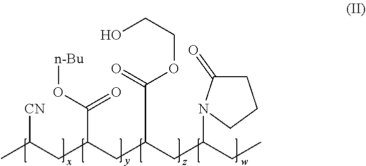

in which, w, x, y and z are as defined above for formula (I),
with an excess of carbonyldiimidazole, optionally in the presence of a solvent;
b) reacting the compound obtained in step a) with an excess of propargylamine, optionally in the presence of a solvent.

Step a) is carried out in the presence of an excess of carbonyldiimidazole. By “excess of carbonyldiimidazole” is meant an amount of between approximately 1.1 and approximately 6 equivalents (based on 1 equivalent of compound (II)).

Compounds of formula (II) can be obtained, for example, as described in U.S. Pat. No. 4,915,755.

The compound obtained at the end of step a) is an “intermediate” compound of formula:

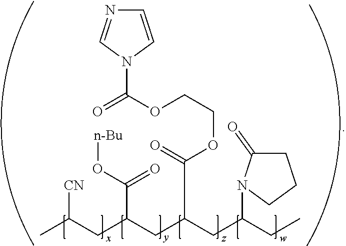

in which, w, x, y and z are as defined above for formula (I), which compound is used as such for carrying out step b).

Step b) is carried out in the presence of an excess of propargylamine. By “excess of propargylamine” is meant an amount of between about 1.5 and about 6 equivalents (based on the intermediate obtained at the end of step a)).

In some embodiments, step a) is carried out at a temperature between room temperature and about 40° C., beneficially at a temperature between room temperature and about 30° C.

In some embodiments, the duration of step a) is between about 15 minutes and about 10 hours, beneficially between about 15 minutes and about 3 hours.

In some embodiments, step b) is carried out at a temperature of between room temperature and about 40° C., beneficially at a temperature of between room temperature and about 30° C. In some embodiments, the duration of step b) is between about 1 hour and about 48 hours, beneficially between 1 hour and about 24 hours.

In some embodiments, step a) is carried out in the presence of a solvent. In practice, it is possible to use a conventional organic solvent, with the exception of nucleophilic solvents. Beneficially, the solvent may be chosen from methylene chloride, chloroform, 1,2-dichloroethane or ethyl acetate.

In some embodiments, step b) is carried out in the presence of a solvent. In practice, it is possible to use a conventional organic solvent, with the exception of nucleophilic solvents. Beneficially, the solvent may be chosen from methylene chloride, chloroform, 1,2-dichloroethane or ethyl acetate.

In some embodiments, the solvent used in step a) and the solvent used in step b) are identical.

The process according to the present disclosure has the following benefits:
it is a one-pot synthesis;
it gives quantitative yields;
it uses low-toxicity and low-cost reagents;
it allows easy purification of the final product.

According to another aspect, the present disclosure relates to compounds of formula (I) obtainable by the process described above. These compounds are useful as neutral polymeric binding agents for the preparation of energetic materials such as composite solid propellants.

The invention will be better understood with the aid of the following example, given by way of illustration. In this example, the number average molar mass (Mn) and mass average molar mass (Mp) of the compound of formula (I) were determined by steric exclusion chromatography (SEC) using the following apparatus:
Equipment: Agilent Infinity 1260 thermostated at 70° C.;
Detection: refractometry;
Eluent: Dimethylsulfoxide, 1.0 ml/min;
Standard: Polymethyl methacrylate.

The polydispersity D of the compound of formula (I) is equal to the ratio Mp/Mn.

The compound of formula (I) was also characterised by NMR using a Bruker AC-400 apparatus.

### Abbreviations

AIBN=azobisisobutyronitrile
CDI=carbonyldiimidazole
DCM=dichloromethane

### Example 1

### Synthesis of a Compound of Formula:

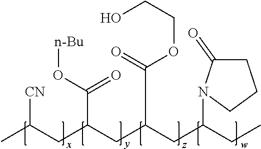

wherein x=1; y=0.3; z=0.2; w=0.1

A 100 mL three-neck round bottom flask was fitted with a thermometer, a condenser and mechanical stirring (300 rpm). It was then charged with 6 mL of acetone and immersed in a water bath at room temperature. Acrylonitrile (4 mL, 60.3 mmol), n-butyl acrylate (2.58 mL, 18.1 mmol), 2-hydroxyethyl acrylate (1.39 mL, 12.1 mmol), N-vinylpyrrolidone (640 μL, 6.0 mmol) and mercaptoethanol (240 μL, 3.38 mmol) were added in portions using syringes. AIBN (206.38 mg, 1.26 mmol, 1.3 mol %) was then added and the residues rinsed with a further 1 mL of acetone. The water bath temperature was then set at 64° C. The reaction continued for 6 h at 60° C., then methanol (20 mL) was added to stop the reaction. The medium was then recovered with acetone and evaporated under reduced pressure to leave only the copolymer. The copolymer was then dried overnight in a deep vacuum (10⁻² mbar) at room temperature. A yellow solid (6.88 g, 85%) was obtained.

¹H NMR (acetone d₆, 400 MHz, 21° C.): δ (ppm)=4.23 (s(br), —OCH₂CH₂OH), 4.14 (s(br), —OCH₂CH₂CH₂CH₃), 3.78 (s(br), —OCH₂CH₂OH), 3.58-2.47 (m, CH_{main chain}), 2.45-1.72 (m, CH_{2 main chain}), 1.58 (s(br), —OCH₂CH₂CH₃), 1.35 (s(br), —OCH₂CH₂CH₍₃₎), 0.88 (s(br), —OCH₂CH₂CH₍₃₎).

ATR-FTIR: ν (cm⁻¹)=3505 (w(b)), 2959 (m), 2935 (m), 2874 (w), 2242 (w), 1725 (s), 1670 (m), 1452 (m), 1267 (m), 1226 (m), 1166 (s), 1067 (m), 936 (w), 891 (w), 842 (w), 739 (w), 652 (w).

SEC (DMSO, refractive index, PMMA calibration): Mₚ=10,600 g·mol⁻¹; Mₙ=25,400 g·mol⁻¹; Ð=2.4.

### Example 2

### Synthesis of a Compound of Formula:

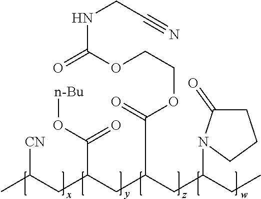

wherein x=1, y=0.3, z=0.2 and w=0.1.

A 50 mL three-neck round bottom flask was fitted with a thermometer, a refrigerant and magnetic stirring (300 rpm). It was then charged with 10 mL of dichloromethane and 1 g of a binding agent of the formula below, and immersed in a water bath at room temperature:

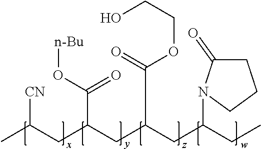

where x=1, y=0.3, z=0.2 and w=0.1.

CDI (365.88 mg; 2.26 mmol; 1.5 equivalents) was then added in one portion and the reaction continued at room temperature for 3 h. Propargylamine (300 μL; 4.72 mmol; 3 equivalents) was then added in one portion and the reaction continued at room temperature for 18 h. At this stage, proton NMR of the reaction medium showed full conversion. The reaction medium was diluted with 40 mL dichloromethane and the organic phase was washed with 3 portions of 50 mL distilled water. It was then dried over magnesium sulfate and evaporated under reduced pressure to recover a yellow solid (1.31 g; Quantitative).

¹H NMR (acetone d₆, 400 MHz, 21° C.): δ (ppm)=6.69 (s(br), NH), 4.33 (s(br), —OCH₂CH₂O—), 4.15 (s(br), —OCH₂CH₂CH₂CH₃), 3.95 (s(br), —CH₂C≡CH), 3.58-2.53 (m, CH_{main chain}), 2.68 (s(br), —CH₂C≡CH), 2.40-1.75 (m, CH_{2 main chain}), 1.66 (s(br), —OCH₂CH₂CH₂CH₃), 1.42 (s(br), —OCH₂CH₂CH₂CH₃), 0.95 (s(br), —OCH₂CH₂CH₂CH₃).

ATR-FTIR: ν (cm⁻¹)=3368 (w), 3282 (w), 2960 (m), 2931 (m), 2874 (w), 2242 (w), 1720 (s), 1677 (m), 1520 (m), 1452 (m), 1239 (s), 1168 (s), 1058 (m), 944 (w), 776 (w), 652 (m).

SEC (DMSO, refractive index, PMMA calibration): Mₚ=12,600 g·mol⁻¹; Mₙ=7,000 g·mol⁻¹; Ð=1.8.

1. A process for the synthesis of a compound of formula (I): 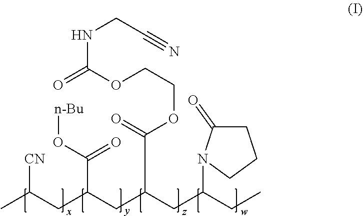
wherein x=1; 0<w≤1; 0<y≤1 and 0<z≤1;
the process comprising:
a) reacting a compound of formula (II): 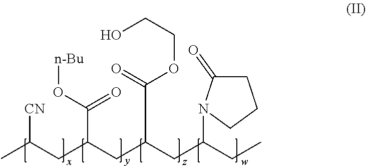
in which, w, x, y and z are as defined above for formula (I),
with an excess of carbonyldiimidazole, optionally in the presence of a solvent;
b) reacting the compound obtained in step a) with an excess of propargylamine, optionally in the presence of a solvent.

2. The process of claim 1, wherein step a) is carried out at a temperature of between about room temperature and about 40° C.

3. The process of claim 1, wherein the duration of step a) is between about 15 minutes and about 10 h.

4. The process of claim 1, wherein step a) is carried out in the presence of a solvent.

5. The process of claim 1, wherein step b) is carried out at a temperature of between about room temperature and about 40° C.

6. The process of claim 1, wherein the duration of step b) is between about 1 h and about 48 h.

7. The process of claim 1, wherein step b) is carried out in the presence of a solvent.

8. The process of claim 1, wherein steps a) and b) are carried out in the presence of a solvent, and the solvent used in step a) and the solvent used in step b) are identical.

9. A compound of formula (I) obtainable by the process of claim 1: 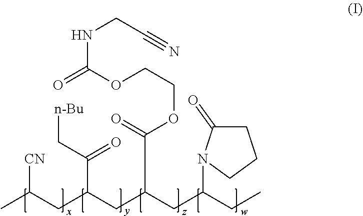
wherein x=1; 0<w≤1; 0<y≤1 and 0<z≤1.
